(19) United States
(12) Patent Application Publication
(10) Pub. No.: US 2018/0248086 A1
(43) Pub. Date: Aug. 30, 2018

(54) DEEP ULTRAVIOLET LIGHT SOURCE AND PACKAGING METHOD THEREFOR

(71) Applicant: QINGDAO JASON ELECTRIC CO., LTD., Qingdao, Shandong Province (CN)

(72) Inventors: Shengmao BAI, QINGDAO, SHANDONG PROVINCE (CN); Jie WANG, QINGDAO, SHANDONG PROVINCE (CN)

(73) Assignee: QINGDAO JASON ELECTRIC CO., LTD., Qingdao, Shandong Province (CN)

(21) Appl. No.: 15/559,359

(22) PCT Filed: Mar. 17, 2016
(86) PCT No.: PCT/CN2016/076559

§ 371 Date: Sep. 18, 2017

(30) Foreign Application Priority Data
Mar. 18, 2015 (CN) 201510117640.5

(51) Int. Cl. H01L 33/54; H01L 33/56; H01L 33/62; H01L 33/64; A61L 2/10

(52) U.S. Cl. CPC H01L 33/54; H01L 33/56; H01L 33/62; H01L 2933/0075; A61L 2/10; H01L 2933/005; H01L 2933/0066; H01L 33/647

(57) ABSTRACT

The invention discloses a deep ultraviolet light source and a packaging method thereof. The deep ultraviolet light source includes a deep ultraviolet light emitting diode chip and a lead frame, the deep ultraviolet light emitting diode chip is fixed on the lead frame, the outside of the deep ultraviolet light source is provided with a transparent protective special layer, the transparent protective special layer forms a convex structure on an top side surface of the deep ultraviolet light emitting diode chip, and the transparent protective special layer forms a recessed structure on the side surface of the deep ultraviolet light emitting diode chip. The convex transparent protective special layer formed on the top side surface of the deep ultraviolet light emitting diode chip and the recessed transparent protective special layer formed on the side can effectively reduce the deep UV loss caused by the total reflection on the side surface and the front top side surface, thereby improving the deep UV radiation level and the UV light source light transmission efficiency; in addition, transparent protective special layer is used to replace the quartz lens and protect the deep ultraviolet light diode and that effectively reduces the cost of packaging.

### TECHNICAL FIELD

The present invention relates to an ultraviolet light source, and more particularly to a deep ultraviolet light source and the packaging method thereof.

### BACKGROUND TECHNOLOGY

The disinfection effect of UV on bacteria and virus is generally taken within a few seconds, while it takes more than 20 minutes to achieve the same disinfection effect with traditional chlorine or ozone disinfection. Among all of the disinfection methods, UV has a broadest spectrum of sterilization to perform more efficiently in killing almost all kinds of bacteria and virus. However, there is a lack of the UV light source. Normally, a mercury lamp is widely used as the UV light source. With the development of technology, deep ultraviolet light-emitting diode has better qualities in low power consumption, long service life and no pollution. Compared to the mercury lamp, the wavelength of the deep ultraviolet light can be adjusted through the semiconductor material production process. Through the process of adjusting the wavelength, scientists found that deep ultraviolet light can be used in many other fields, such as skin disease treatment or military remote sensing, and the like. As shown in FIG. 1, a deep ultraviolet light source made mainly by a deep ultraviolet light emitting diode in the prior art generally encapsulates the deep ultraviolet light emitting diode 101 into the metal-tube shell 102, and a quartz lens 103 is provided above the metal-tube shell 102 to adjust the light emitting angle. But the use of the quartz lens 103 causes a serious loss of the ultraviolet light generated by the deep ultraviolet light emitting diode 101 and a high packing cost. How to design a deep ultraviolet light source with high light transmission efficiency and low packaging cost are the technical problems to be solved by the present invention.

### SUMMARY OF THE PRESENT INVENTION

The present invention provides a deep ultraviolet light source and a packaging method thereof to improve the light transmission efficiency of the deep ultraviolet light source and reduce the packaging cost.

In order to solve the above-mentioned problems, the present invention is implemented by the following technical solution.

A deep ultraviolet light source comprises a deep ultraviolet light emitting diode chip and a lead frame, the deep ultraviolet light emitting diode chip is fixed on the lead frame, the outside of the deep ultraviolet light emitting diode chip is provided with a transparent protective special layer, the transparent protective special layer forms a convex structure on the top side surface of the deep ultraviolet light emitting diode chip, and the transparent protective special layer forms a recessed structure on the side surface of the deep ultraviolet light emitting diode chip.

Further, a mounting groove is formed on the lead frame, the deep ultraviolet light emitting diode chip is arranged in the mounting groove, an annular space is formed between the side surface of the deep ultraviolet light emitting diode chip and the side surface of the mounting groove, and the transparent protective special layer forms the recessed structure in the annular space.

Further, the lead frame is provided with two electrode terminals, and the electrodes of the deep ultraviolet light emitting diode chip are connected to the corresponding electrode terminals, respectively.

Further, a thermal sink is further provided between the deep ultraviolet light emitting diode chip and the lead frame.

Further, the transparent protective special layer is made of resin or silica gel.

The present invention also provides a packaging method of the deep ultraviolet light source to encapsulate the deep ultraviolet light source mentioned above, comprising the following steps: placing the deep ultraviolet light emitting diode chip on the lead frame and dropping liquid transparent medium on top side surface of the deep ultraviolet light emitting diode; liquid transparent medium is slowly infiltrated to the side surface of the deep ultraviolet light emitting diode chip and to the lead frame by the force of gravity; the convex structure is formed on the top side surface of the deep ultraviolet light emitting diode chip and the recessed structure of the transparent protective special layer is formed on the side surface of the deep ultraviolet light-emitting diode chip by the force of surface tension.

Compared with the prior art, the advantages and the positive effects of the invention are: in the deep ultraviolet light source and the packaging method provided by the present invention, a convex transparent protective special layer formed on the top surface of the deep ultraviolet light emitting diode chip and a recessed transparent protective special layer formed on the side can effectively reduce the deep UV loss caused by the total reflection on the side surface and the top face, thereby improving the deep UV radiation level and the UV light source light transmission efficiency. In addition, transparent protective special layer is used to replace the quartz lens and protect the deep ultraviolet light diode and that effectively reduces the cost of packaging.

The above and other features and advantages of the present invention will be more apparent by describing in detail exemplary embodiments thereof with reference to the accompanying drawings.

### BRIEF DESCRIPTION OF THE DRAWINGS

The embodiments of the present invention or the technical solutions of the prior art will be more apparent by describing briefly the drawings which are referred in illustrating the embodiments or the prior art. Obviously, the drawings described below are only part examples, a person of ordinary skill in the art will understand and appreciate that various modifications can be made without departing the scope of the present disclosure, in which:

FIG. 1 is a schematic illustration of a conventional deep ultraviolet light source in packaging;

FIG. 2 is one schematic diagram of the embodiment of the deep ultraviolet light source of the present invention;

FIG. 3 is another schematic diagram of the embodiment of the deep ultraviolet light source of the present invention.

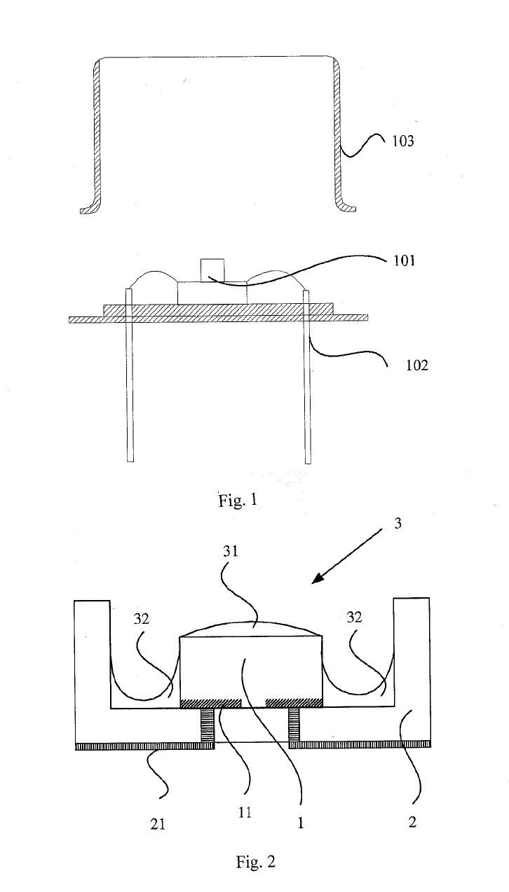

### EMBODIMENTS OF THE PRESENT INVENTION

The above and other objects, technical solutions and advantages of the present disclosure will become more apparent and completed to those of ordinary skill in the art by describing in detail exemplary embodiments thereof with reference to the accompanying drawings. Embodiments and features as described and illustrated in the present disclosure are only preferred examples, and various modifications thereof may also fall within the scope of the disclosure.

As shown in FIG. 2, the deep ultraviolet light source of the present embodiment comprises a deep ultraviolet light emitting diode 1 and a lead frame 2. The deep ultraviolet light emitting diode 1 is fixed on the lead frame 2, and the outside of the deep ultraviolet light emitting diode 1 is provided with a transparent protective special layer 3. The transparent protective special layer 3 forms a convex structure 31 on an top side surface of the deep ultraviolet light emitting diode 1, and the transparent protective special layer 3 forms a recessed structure 32 on the side surface of the deep ultraviolet light emitting diode chip 1.

Specifically, the deep ultraviolet light emitting diode chip 1 in the deep ultraviolet light source of the present embodiment is fixed on the lead frame 2 to protect the deep ultraviolet light emitting diode chip 1. The transparent protective special layer 3 provided on the outside of the deep ultraviolet light emitting diode chip 1 is used to replace the quartz lens of the prior art, wherein the transparent protective layer 3 forms the convex structure 31 and the recessed structure 32 on the top side surface and the side surface of the deep ultraviolet light emitting diode chip 1 respectively. During the luminescence of the deep ultraviolet light emitting diode chip 1, the convex surface of the convex structure 31 narrows the incident angle, and the light emitted from the deep ultraviolet light emitting diode chip 1 can be refracted into the air to increase the luminous power by the convex structure 31. Moreover, the concave surface of the recessed structure 32 also narrows the incident angle on the side of the deep ultraviolet light emitting diode chip 1, and the light emitted by the deep ultraviolet light emitting diode chip 1 can be refracted into the air by the recessed structure 32 to further increase the luminous power.

It is preferable that a mounting groove (not shown) is formed on the lead frame 2, and the deep ultraviolet light emitting diode chip 1 is arranged in the mounting groove, and an annular space (not shown) is formed between the side surface of the deep ultraviolet light emitting diode chip 1 and the side surface of the mounting groove 2. The transparent protective layer 3 forms the convex structure 31 in the annular space. In particular, by forming the recessed structure 32 in the annular space, the curvature of the concave surface formed by the recessed structure 32 can be effectively increased so that the incident angle on the side is less than the critical angle, thereby further increasing the light emitting rate on the side.

Wherein the lead frame 2 are provided with two electrode terminals 21, and the electrodes 11 of the deep ultraviolet light emitting diode chips 1 are connected to the corresponding electrode terminals 21. In addition, the transparent protective special layer 3 is made of transparent material such as resin or silicone gel.

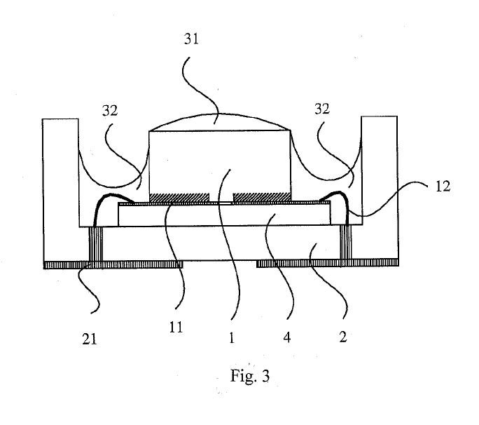

In addition, the deep ultraviolet light emitting diode chip 1 in the present embodiment may be directly fixed on the lead frame 2, or thermal sink may be provided between the deep ultraviolet light emitting diode chip 1 and the lead frame 2 as shown in FIG. 3

In this embodiment, the deep ultraviolet light source is formed by forming a transparent protective special layer on the end surface of the deep ultraviolet light emitting diode chip and forming a recessed transparent protective layer on the side surface so as to effectively reduce the loss of the deep ultraviolet ray caused by the total reflection on the side surface and the front top side surface, thereby improving the UV radiation level and UV light source light transmission efficiency. In addition, transparent protective special layer is used to replace the quartz lens and to protect the deep ultraviolet light diode that effectively reduces the cost of packaging.

The packaging method of the deep ultraviolet light source of the present embodiment is characterized in that specifically comprises the following steps: placing the deep ultraviolet light emitting diode chip on the lead frame and dropping liquid transparent medium on the top side surface of the deep ultraviolet light emitting diode chip; liquid transparent medium is slowly infiltrated to the side of the deep ultraviolet light emitting diode chip and to the lead frame under the force of gravity; the convex structure is formed on the top side surface of the deep ultraviolet light emitting diode chip and the recessed structure of the transparent protective special layer is formed on the side surface of the deep ultraviolet light-emitting diode under the force of surface tension.

It should be understood that the above embodiments are merely illustrative of the technical solutions of the present invention and are not intended to be limitation thereof. While the invention has been described in detail with reference to the foregoing embodiments, it will be understood by one of ordinary skill in the art that it may still be modified or equivalently replaced by some of the technical features thereof, and these modifications or substitutions do not depart from the spirit and scope of the technical solutions of the various embodiments of the present invention.

1. A deep ultraviolet light source, comprising a deep ultraviolet light emitting diode chip and a lead frame, the deep ultraviolet light emitting diode chip is fixed on the lead frame, a transparent protective special layer is provided at outside of the deep ultraviolet light source, the transparent protective special layer forms a convex structure on a top side surface of the deep ultraviolet light emitting diode chip, and the transparent protective special layer forms a recessed structure on a side surface of the deep ultraviolet light emitting diode chip.

2. The deep ultraviolet light source of claim 1, wherein a mounting groove is formed on the lead frame, the deep ultraviolet light emitting diode chip is arranged in the mounting groove, an annular space is formed between the side surface of the deep ultraviolet light emitting diode chip and the side surface of the mounting groove, and the transparent protective special layer forms the recessed structure in the annular space.

3. The deep ultraviolet light source of claim 1, wherein the lead frame is provided with two electrode terminals, and the electrodes of the deep ultraviolet light emitting diode chip are correspondingly connected to the electrode terminals.

4. The deep ultraviolet light source of claim 1, wherein a thermal sink is further provided between the deep ultraviolet light emitting diode chip and the lead frame.

5. The deep ultraviolet light source of claim 1, wherein the transparent protective special layer is made of resin or silica gel.

6. A packaging method to encapsulate the deep ultraviolet light source of claim 1, comprising the following steps: placing the deep ultraviolet light emitting diode chip on the lead frame and dropping liquid transparent medium on the top side surface of the deep ultraviolet light emitting diode chip; slowly infiltrating a liquid transparent medium to the side surface of the deep ultraviolet light and to the lead frame under the force of gravity; forming the convex structure on the top side surface of the deep ultraviolet light emitting diode chip and forming the recessed structure of the transparent protective special layer on the side surface of the deep ultraviolet light-emitting diode under the force of surface tension.
